(19) United States
(12) Patent Application Publication
(10) Pub. No.: US 2021/0325390 A1
(43) Pub. Date: Oct. 21, 2021

(54) Virus detection

(71) Applicant: Michael L. Bell, Fullerton, CA (US)

(72) Inventor: Michael L. Bell, Fullerton, CA (US)

(21) Appl. No.: 17/233,479

(22) Filed: Apr. 17, 2021

Related U.S. Application Data
(60) Provisional application No. 63/011,939, filed on Apr. 17, 2020.

(51) Int. Cl. G01N 33/569; G01N 33/58; G01N 33/543; G01N 21/76; G01N 15/14

(52) U.S. Cl. CPC G01N 33/56983; G01N 33/581; G01N 2446/00; G01N 21/76; G01N 15/1404; G01N 33/54326

(57) ABSTRACT

The invention includes methods and kits for the detection of a virus that may be in a sample. In embodiments, the invention includes a method of detecting a virus in a sample by forming a reaction mixture exposing an aliquot of the sample purportedly containing a virus to an immobilized receptor. A soluble receptor including a detectable label is added to the reaction mixture. A washing step washes the immobilized receptor, and the detectable label is detected.

### TECHNICAL FIELD

This invention relates to methods and kits for detecting viruses.

### BACKGROUND ART

Coronaviruses (CoVs) (order Nidovirales, family Coronaviridae, subfamily Coronavirinae) are enveloped viruses with a positive sense, single-stranded RNA genome. The novel coronavirus SARS-CoV-2, causative agent of COVID 19 disease, is detectable by a nucleic acid (NA) assay such as real time or quantitative polymerase chain reaction (rtPCR or qPCR) after reverse transcription of a segment of viral RNA or by a similar NA amplification assay. Such NA amplification assays have high specificity and potentially high sensitivity, though the sensitivity is not always realized due to sample processing limitations. However, NA amplification assays are complex and expensive. The expense is in part due to the mechanics of amplification where positive samples produce amplification products that could contaminate negative samples. The susceptibility to amplification product contamination generally requires single use consumables, a major contributor to costs. These consumables have high direct costs and need to be handled either by an operator, increasing labor costs, or by an automated system, increasing system costs.

Further, NA amplification assays generally are multiple-step procedures including sample preparation steps to free NAs from samples and amplification and detection steps that often include thermal cycling. Each of these adds to costs through more complex systems or consumables.

Still further, most NA amplification assays rely on materials that must remain refrigerated. This further contributes to costs both for systems and for storage of consumables.

Because of their complexity, NA amplification assays often are time consuming, in part because samples may be retained for batch processing, which can improve cost per test. Some point of care systems feature rapid turnaround times for single samples at the cost of complex consumables and manual loading. There is thus a need for a viral assay that improves on these cost and time limitations.

A response to the COVID 19 pandemic has been to limit transmission through social distancing. This has had the side effect of shutting down large portions of the economy. An ultimate solution to the pandemic is effective vaccination or “herd” immunity. Alternatively, until effective vaccination is fully implemented, each detected case may be isolated so that the possibility of transmission is eliminated. This requires widespread, frequent, and low-cost testing of exposure. There is thus a need for a viral assay that provides the capability for widespread, frequent, and low-cost testing.

Some viruses can survive on surfaces or in aerosols for hours or days. Exposure to these surfaces or aerosols may later infect exposed individuals even if social distancing guidelines are followed. Disinfection of surfaces can help, but ideally it must be done after each possible exposure. There is thus a need to determine whether surfaces or locations are contaminated. Plainly, cost is critical since frequent retest is likely.

Coronaviruses such as SARS-CoV-2 are RNA viruses, which rely on ribonucleic acid (RNA) to store and transmit genetic information. NA amplification assays either directly or indirectly amplify portions of this RNA to detect the virus. Ribonucleases (RNase) that degrade RNA are ubiquitous in living organisms including humans. Some cells secrete copious quantities of non-specific RNases. RNases are, therefore, extremely common, resulting in very short lifespans for any RNA that is not in a protected environment. While this is beneficial in protecting organisms against RNA viruses, these enzymes can destroy the viral RNA that forms the basis of detection in NA amplification assays. As a result, great care must be taken in collecting and transporting specimens for SARS-CoV-2 assays to isolate them from environmental and technician-produced RNase. There is thus a need for a viral assay that is insensitive to the presence of RNase.

Individuals who are apparently asymptomatic may spread the virus, and the ubiquity of travel allows transmission to exposure-naive persons, particularly in close quarters such as cruise ships or assemblies. There has been some effort to screen out the infected such as by excluding those with fevers because this may be measured very quickly and inexpensively. However, the large fraction of asymptomatic carriers makes this problematic at best. There is thus a need for a rapid, low-cost monitoring assay and system to screen out infective carriers.

NA amplification assays are paradoxically the easiest assays to establish for a novel virus despite their complexity. Once an organism is sequenced, any laboratory can get access to the required NA components (primers, probes, etc.) without significant development effort. In contrast, an immunoassay generally needs an extended time and access to viral components to develop antibodies. Once developed, immunoassays are generally lower cost but have lower specificity than NA amplification assays. There is thus a need for an assay that can be rapidly deployed following the characterization of a new virus.

Widespread infections, such as the COVID 19 pandemic, make likely the evolution of variants which are more infectious. Some of these variants may also cause more severe illness or may evade immune responses derived from earlier infection or vaccination. The US CDC has defined three classifications for SARS-CoV-2 variants including Variants of Interest (VOI), Variants of Concern (VOC), and Variants of High Consequence (VOHC). As of the date of filing, five VOCs have been identified in the US. One of them, designated 6.1.1.7, first identified in the UK in late 2020, has outcompeted earlier strains to dominate new US cases.

While B.1.1.7 is apparently not substantially more virulent than earlier strains and responds well to immunity produced by first generation vaccines, this may be less true of other variants. B.1.351 was first identified in South Africa in December 2020 and is apparently both more virulent and more resistant to vaccine-derived immunity.

P.1, B.1.427, and B.1.429 have also been designated as VOCs. New variants are undoubtably generated every day though most do not have transmission advantages over preexisting strains.

The newer variants have been identified by NA sequencing of isolates from identified cases of COVID 19. Since some COVID 19 cases are asymptomatic, and most COVID 19 cases are identified by NA testing based on known sequences, it is possible that new variants may arise that are not detectable by existing NA assays. Some variants may also evade detection by immunoassays as the change in NA sequence in the variants can produce changes in the target antigens bound in immunoassays.

There is thus a need for an assay that can detect variants of a virus that may be different from that originally targeted by other assays. There is also a need for a functional assay that that is capable of detecting infectious viral variants independently of the viral NA sequence.

### DISCLOSURE OF INVENTION/SUMMARY

The invention includes methods and kits for the detection of a virus that may be in a sample.

In embodiments, the invention includes a method of detecting a virus in a sample by forming a reaction mixture exposing an aliquot of the sample purportedly containing a virus to an immobilized receptor. A soluble receptor including a detectable label is added to the reaction mixture. A washing step washes the immobilized receptor, and the detectable label is detected. The term receptor as used herein means a version of the “natural” target of a virus as opposed to, for example, an antibody.

The virus may be a coronavirus, such as a variant of SARS-CoV-2.

In some embodiments, the washing step takes place under controlled stringency conditions.

The immobilized receptor may include at least a portion of an angiotensin converting enzyme 2 (ACE2). Embodiments of the immobilized receptor include a receptor bound to any of a magnetic particle, a paramagnetic particle, a superparamagnetic particle, a dye-labeled particle, a porous substrate, a solid substrate, a gel, or a fluid interface. In some embodiments, the solid substrate includes a wall of a container.

The soluble receptor may comprise at least a portion of an ACE2. The detectable label may include one or more of a dye, a fluorescent marker, a quantum dot, a mass tag, an enzyme, a nucleic acid, a particle, a magnetic tag, a radionuclide, or a colloidal metal.

In other embodiments, one of the immobilized receptor or the soluble receptor may be replaced by another specific binding molecule, such as a specific antibody. This has the benefit of allowing simultaneous incubation with both immobilized binder and labeled binder if the specific binding molecule has specificity different than the receptor.

In embodiments, the washing step includes exposing the immobilized receptor to a wash liquid. The washing step may include controlled stringency conditions comprising control of one or more of a temperature, a pH, an ionic strength, a hydrophobicity, a chaotrope concentration, a surfactant concentration, a competitor concentration, a volume, or a flow rate of the wash liquid. The ionic strength of the wash liquid may be less than about 0.10 mol/kg.

In some embodiments, the method may also include steps of forming a second reaction mixture by exposing a second aliquot of the sample to a second immobilized receptor and adding a second soluble receptor including a second detectable label to the second reaction mixture. The second immobilized receptor may be washed, and a second signal detected from the second detectable label. The signal from the detectable label may then be compared to the second signal from the second detectable label to determine the presence of the virus. The step of washing the second immobilized receptor may occur under different stringency conditions than the step of washing the immobilized receptor.

The coronavirus may be SARS-CoV-2; the immobilized receptor and the soluble receptor may each include at least a portion of an ACE2.

In embodiments, the immobilized receptor includes a superparamagnetic particle, and the detectable label includes an enzyme capable of acting on a chemiluminescent substrate to produce light. In other embodiments, the immobilized receptor includes a lateral flow substrate. In still other embodiments, the immobilized receptor includes a particle and the step of detecting the signal includes characterizing the particle in a flow cytometer.

The invention also includes kits that have a lateral flow substrate, an ACE2 immobilized to the lateral flow substrate, a developing solution including an ACE2 coupled to a detectable label, and a first wash liquid. The first wash liquid may be configured to displace at least 50% of a SARS CoV coronavirus from the substrate but less than 50% of a SARS CoV-2 coronavirus from the substrate.

In embodiments, the first wash liquid may have an ionic strength of less than about 0.15 mol/kg. The first wash liquid may alternatively have an ionic strength of less than about 0.10 mol/kg. The first wash liquid may additionally or alternatively have a pH greater than about 8.2 or less than about 5.1.

In other embodiments, the invention includes kits having an immobilized ACE2, an ACE2 coupled to a detectable label, together with a first wash liquid having an ionic strength less than about 0.10 mol/kg and a second wash liquid having an ionic strength greater than about 0.10 mol/kg.

### BRIEF DESCRIPTION OF THE DRAWINGS

None

### DETAILED DESCRIPTION

Viral threats arise through evolution of viruses to more virulent or more contagious forms and their exposure to human populations. SARS-CoV-2 (the causative agent of COVID 19) is less virulent than SARS-CoV (the causative agent of SARS) but more contagious; this enables a rapid spread that results in more morbidity and mortality. One feature of SARS-CoV-2 is its higher affinity (by a factor of ten to twenty according to published accounts) of the viral spike protein to the human angiotensin-converting enzyme 2 receptor (ACE2). This invention takes advantage of this viral evolution.

Spike proteins give coronaviruses their characteristic appearance of a roughly spherical particle with a “crown” of proteins extending from the main body of the particle, but other viral types also include multiple binding moieties that are associated with human receptors and infection.

Human ACE2 is a membrane-bound aminopeptidase responsible for the production of vasodilatory peptides such as angiotensin. Human ACE2 has been cloned, perhaps in part because a soluble receptor is a possible therapeutic agent. Recombinant human ACE2 expressed in human HEK 293 cells is commercially available from Millipore Sigma of St. Louis, Mo.. The product is a C-terminally flagged and his-tagged glycoprotein with a calculated molecular mass of 85.9 kDa (amino acids GIn18-Ser740, with a C-terminal 10-His tag). The human cells expression system allows human-like glycosylation and folding; the relatively high affinity of SARS-CoV-2 spike proteins persists for this cloned variant. Recombinant Human ACE-2 from R&D Systems of Minneapolis, Minn. is prone to proteolytic cleavage at C-terminus according to the manufacturer. Tai et al. (cited below) established that this soluble form of ACE2 was able to block spike protein receptor-binding domain from binding to ACE2 on cells.

Native ACE2 is a single-pass type I membrane protein, with its enzymatically active domain exposed on the surface of cells in lungs and other tissues. A soluble form of ACE2, lacking the cytosolic and transmembrane domains, has been shown to block binding of the SARS-CoV spike protein to ACE2 on cultured cells. This is consistent with the concept that coronaviruses recognize and bind to the extracellular domain, which is conserved in the soluble form. For convenience, I will designate the soluble form as solACE2, recognizing that more than one soluble form may exist or be producible. In this document solACE2 refers to a soluble form of ACE2, and any labeled analogs, that retain the ability to bind SARS-CoV-2 spike protein with relatively high affinity.

The SARS-CoV-2 spike protein has a high relative affinity for human ACE2 as compared to that of SARS-CoV as described by W Tai et al. in Characterization of the receptor-binding domain ( RBD) of 2019 novel coronavirus: implication for development of RBD protein as a viral attachment inhibitor and vaccine (published online ahead of print, 2020 Mar. 19, Cell Mol Immunol. 2020;10.1038/s41423-020-0400-4. doi:10.1038/s41423-020-0400-4). Tai et al. measured the binding between SARS-CoV-2 RBD and hACE2-expressing 293T cells. This was much stronger (EC₅₀ of 0.08±0.02 μg/mL) than the binding between SARS-CoV RBD and these cells (E₅₀ of 0.96±0.01 μg/mL). Further, Tai et al. also found stronger binding between SARS-CoV-2 RBD and soluble hACE2 (EC₅₀ of 1.07±0.10 μg/mL) as compared to SARS-CoV RBD and soluble ACE2 (EC50 of 1.66±0.46 μg/mL). Though the binding to soluble hACE2 was of lower affinity than that of intact hACE2 on cells, in both cases SARS-CoV-2 bound the enzyme more tightly than did SARS-CoV. The term hACE2 refers to human ACE2; RBD is the receptor-binding domain of the respective spike protein.

Among parameters that quantitatively indicate the affinity of a ligand for a receptor are the binding affinity (ΔG) and the equilibrium dissociation constant (Kd). J T Ortega, et al. in Role of changes in SARS-CoV-2 spike protein in the interaction with the human ACE2 receptor: An in silico analysis. (EXCLI J. 2020;19:410-417. Published 2020 Mar. 18. doi:10.17179/excli2020-1167) calculated the binding affinity (ΔG) and dissociation constant (Kd) at 25 Celsius of the binding of human ACE2 by the spike proteins of SARS-Cov and SARS-CoV-2 viruses based on the crystal structure of the proteins. For SARS-CoV, AG was calculated as −14.1 kcal/mol and Kd as 1.1×10⁻¹⁰M. For SARS-CoV-2, ΔG was calculated as −15.7 kcal/mol and the Kd was 5×10⁻¹²M. These values confirm the measured substantially greater affinity of SARS-CoV-2 spike protein for ACE2 as compared to SARS-CoV.

Washing in an assay is generally a process by which unbound or lightly bound materials are removed from a solid phase. Usually the goal is to remove nonspecifically bound materials and retain those that are specifically bound, such as by an antibody. A wash process may also be employed to remove specifically bound material. A more stringent wash is one that removes more tightly bound material. In the context of a plurality of possible receptor-ligand interactions, a stringent wash will remove some ligands that are bound, even though those ligands are bound specifically.

The concept of controlled stringency washing is well-developed for NA hybridization. NA duplexes have consistent binding forces depending on the length of the oligos and the match between the bases at each position. The change in binding affinity and effect of changes in wash stringency (at least with regard to wash liquid temperature) are now substantially predictable in nucleic acid hybridization interactions.

In protein-protein interactions, each pair of proteins is generally a new adventure; there is no simple relationship between binding conditions and binding forces because the interaction is governed primarily by secondary and tertiary structures of the participating molecules. However, when crystal structures of the molecules are known with high precision, modeling using molecular docking based on these structures and on the nature of the interacting residues and the type of chemical interactions occurring between the interacting residues are calculable and predictable. This is true for spike protein ACE2 interaction as confirmed by the correspondence between the calculated Ortega parameters and the Tai measured EC₅₀s of the actual proteins.

Ortega et al. identified 117 individual protein-protein contacts (PPCs) between SARS-CoV and ACE2. They identified 143 such contacts between SARS-CoV-2 and ACE2. The table below lists the number of protein-protein contacts characterized by type of interaction (adapted from Ortega et al.)

| types of contacts (PPCs) with ACE2 | SARS-CoV | SARS-CoV-2 |
|---|---|---|
| charged-charged | 5 | 13 |
| charged-polar | 14 | 13 |
| charged-nonpolar | 28 | 38 |
| polar-polar | 6 | 14 |
| polar-nonpolar | 32 | 33 |
| nonpolar-nonpolar | 32 | 32 |
| Total PPCs | 117 | 143 |

Ortega et al. identified five spike protein residues as key to the interaction between virus and ACE2. Four of these residues were different between the two viruses. All of the changes at these residues are within the same general category of residues (i.e. polar for polar, nonpolar for nonpolar).

Standout differences between the interactions are more than doubling (in favor of SARS-CoV-2) of the number of PPCs that are charged-charged interactions and polar-polar interactions. Polar residues may have significant partial charges and form hydrogen bonds and other less specific electrostatic interactions among themselves and with charged residues. There is also about a 35% increase in PPCs that are charged-nonpolar interactions. Overall, binding of ACE2 by SARS CoV is more dependent on nonpolar interactions than is that of SARS CoV-2.

Charge—solvent interactions and solvent screening of charge—charge interactions significantly affect the electrostatic energies of proteins. Lower ionic strength of a solvent increases the relative effects of electrostatic interactions (charged and polar) through reduced screening producing a longer Debye length. Conversely, high ionic strength favors hydrophobic interactions. Lower ionic strength environment may favor the relative binding of SARS CoV-2 to ACE2 because of its greater reliance of charged and polar residues for binding ACE2. Even at normal physiologic ionic strength, the lower binding energy of SARS CoV results in preferential binding of ACE2 by SARS CoV-2 when ACE2 is limited.

A washing step would be expected to remove a relatively larger amount of ACE2 bound to SARS-CoV than bound to SARS CoV-2 because of the difference in binding energy/affinity. Extended or more stringent washing remove more bound material, with the more loosely bound ligands disproportionately removed. There is no magical degree or stringency of washing that would selectively remove ligand from only one spike protein if both were present.

An optimum degree or stringency of washing maximizes the signal to noise ratio considering the non-target virus as unwanted binding. This optimum depends on details of the system implementation including details of detection, of signal processing, and of the point chosen for the system receiver operation characteristic in the tradeoff between sensitivity and specificity. Determining the optimum conditions and degree of washing is usually an empirical task in assay and system development.

The stringency of wash is adjustable. For example, commercial antibody stripping buffers (usually of extreme pH and frequently including chaotropes such as sodium dodecyl sulfate (SDS)) can remove essentially all antibody specifically bound to proteins in a Western blot. Although intended to preserve the target proteins, some loss cannot be avoided. In the case of coronaviruses, ionic surfactants, strong chaotropes, or high proportions of alcohols or other organic solvents may disrupt the viral envelope. Since this likely destroys the efficacy of the assay, such materials must be carefully tested before inclusion in a washing protocol. Common chaotropic agents include n-butanol, ethanol, guanidinium chloride, lithium perchlorate, lithium acetate, magnesium chloride, phenol, 2-propanol, SDS, thiourea, and urea.

Although there are variations in individual virions, each SARS-CoV-2 includes on the order of 50 to a few hundred spike proteins. This number was determined from analysis of published stained TEM images, which generally show viruses in a narrow focal plane or section as a circle with surrounding spike proteins. The relatively large number of spike proteins per intact virion provides an intrinsic signal amplification, since multiple copies of the detectable label may be attached via solACE2 to each virion. The large number of spike proteins also makes viral capture on a solid phase more efficient as contact between the solid phase and a virion is likely to occur irrespective of the orientation of the virion with respect to the solid phase.

In embodiments, the invention includes assay materials, kits, and methods. The kits include some at least some assay materials. The methods use assay materials to detect a coronavirus in a sample suspected of containing such a virus.

Assay materials include human ACE2 bound to a solid phase. There is an extensive literature describing methods of binding proteins such as ACE2 to a solid phase. These methods depend on the nature of the solid phase (polymer, gel, glass, paper, etc.). These methods are well known in the art.

As a nonlimiting example, methods of binding may include absorption onto hydrophobic polymers such as polystyrene. This may be particularly valuable for intact ACE2 because its hydrophobic transmembrane domain may readily absorb to this hydrophobic surface. Ganged vessels of polystyrene formed as conventional microwell plates can support multiple simultaneous assays once prepared. in other examples, covalent binding may be useful for solid phases with active surface groups, such as superparamagnetic latex particles. Exemplary particles are Dynabeads™ M-280 Tosylactivated available from Invitrogen Corporation of Carlsbad, Calif. These include surface sulphonyl esters that can react covalently with proteins. ACE2 (such as that mentioned above) may be immobilized to these particles according to the manufacturer's directions.

Other assay materials include solACE2 labeled with a detectable label. One form of solACE2 is available from R and D Systems as mentioned above. Other forms may arise from the action of Tumor Necrosis Factor-α Convertase on human cells bearing ACE2 as described by DW Lambert, et al. Tumor necrosis factor-alpha convertase (ADAM17) mediates regulated ectodomain shedding of the severe-acute respiratory syndrome-coronavirus (SARS-CoV) receptor, angiotensin-converting enzyme-2 (ACE2). (J Biol Chem. 2005;280(34):30113-30119. doi:10.1074/jbc.M505111200.) There is an extensive literature describing methods of labeling proteins such as solACE2. These methods vary by the nature of the label and are well known in the art.

As a nonlimiting example, methods of labeling solACE2 include use of Atto 647N Protein Labeling Kit available from Millipore Sigma of St. Louis, Mo. SolACE2 may be labeled with the Atto 647 fluorescent dye using this kit according to the manufacturer's directions.

Another assay material is a washing liquid. Washing liquid may be a simple buffer, such as phosphate buffered saline (PBS) or Tris buffered saline (TBS) with or without an anionic surfactant such as Tween 20 or Triton X 100 at about 0.1% v/v. Such materials (often sold as dry salts for reconstitution and addition of surfactant) are widely available from commercial suppliers such as Millipore Sigma of St. Louis, Mo. In some embodiments, as mentioned above, the washing liquid may be of lower ionic strength to improve the relative affinity of SARS CoV-2 for ACE2 as compared to that of SARS CoV for ACE2. This may be of importance where other coronaviruses might produce false positive results.

Human coronaviruses account for 15 to 30% of common colds, with only occasional spreading to the lower respiratory tract. The coronavirus MERS virus spike protein does not bind to ACE2; it recognizes another receptor DPP4. However, the human coronavirus NL63 also binds to ACE2 but only causes upper respiratory infections. Thus, some other coronaviruses may be present in samples and potentially produce false positive results in a SARS CoV-2 assay as described. The chance of this can be reduced by a more stringent wash as discussed above. A more stringent wash may simply involve an additional volume or cycle of washing. Alternatively, a more stringent wash may include adjusting the wash liquid by lowering the ionic strength (such as to less than 0.10 mol/kg) or by adding small amounts of chaotropic agents such as SDS or bile salts, or by raising the wash temperature to above 37 Celsius, or by including any of the other additives discussed above or a combination of any of these. Washing liquid maybe supplied as a dry material to be hydrated near the time of use.

SARS CoV-2 has the highest known affinity for human ACE2 of characterized coronaviruses. Viruses are continually evolving, and many unknown coronaviruses likely exist. Thus, it is not unreasonable to expect that there may be another coronavirus with comparable or higher ACE2 affinity for human ACE2. While this might introduce a false negative result, it is desirable to identify such hypothetical viruses because of their infectious potential. The described virus detection process may thus serve as an early warning signal of a new infectious agent.

A variation of the described assay can distinguish viruses with different affinities for a common receptor by an “elution” process that measures the signal from the detectable label at two points in a wash sequence (or in two parallel assays with different stringency washes where the label needs subsequent development). If two viruses are present, both would contribute to the signal following the early, less stringent wash. There would be lower contribution of signal from the virus with lower affinity after the later, more stringent wash. The test would likely need to be calibrated to fully separate the signals from each virus.

Still other assay materials are intermediate products of the assay. These intermediate products include a coronavirus immobilized to a solid phase, where the solid phase includes bound ACE2 (or a portion thereof). The coronavirus may be a SARS CoV-2 virus or other coronavirus that has binding affinity for ACE2. Other assay materials include a “sandwich” of a solACE2 having a detectable label, where the solACE2 is specifically bound to a coronavirus that is immobilized to a solid phase, where the solid phase includes bound ACE2. These materials may be produced in the course of the described method by applying a sample containing SARS CoV-2 to the solid phase with immobilized ACE and adding a labeled solACE2.

Kits of the invention include two or more assay materials. These may be packaged with instructions, either human or machine readable, that describe for an operator or a system how the kits should be processed to implement a method. In some embodiments, a kit may include an immobilized receptor, a receptor coupled to a detectable label and two different wash buffers to provide two different stringencies of wash. For example, the low stringency wash buffer may have ionic strength greater than 0.10 mol/kg or greater than about 0.2 mol/kg. The higher stringency wash buffer may have ionic strength less than about 0.1 mol/kg. The kits may also include other ancillary materials. For example, one kit may contain:
a first bottle containing ACE2 receptor immobilized to superparamagnetic particles,
a second bottle containing solACE2 receptor labeled with the enzyme alkaline phosphatase,
a third bottle containing a low stringency wash buffer having ionic strength of more than 0.10 mol/kg,
a fourth bottle containing a high stringency wash buffer having ionic strength of greater than 0.10 mg/kg
a fifth bottle containing 4-methoxy-4-(3-phosphatephenyl)spiro [1,2-dioxetane-3,2′-adamantane], disodium salt. This is a lumogenic substrate for alkaline phosphatase detection sold as Lumigen PPD by Lumigen of Southfield, Mich.

In embodiments, the method includes steps of forming a reaction mixture by combining a specimen with the solid phase immobilized receptor, adding a soluble receptor including a detectable label to the reaction mixture, washing the solid phase, and detecting the detectable label.

The methods resemble those of other solid phase ligand assays, of which sandwich immunoassays are the most common example. There are multiple formats for sandwich assays in the art; the skilled practitioner will recognize those that may be applied to the disclosed assay. These vary with the level of automation and the nature of the solid phase.

In some embodiments, a lateral flow method may be manually operated and use porous solid phases where fluids may be impelled through the porosities by capillary action. However, these may alternatively be operated by mechanical means such as the centrifugal action described in U.S. Pat. No. 5,496,520A to Kelton, Bell, and Chung, incorporated herein by reference. The capture receptors (including ACE2) may be entrapped within or bound to the porous solid phase, with specimen, labeled soluble receptor, and wash liquid sequentially added. Flow of liquids through the porous solid phase and past the immobilized capture receptors provides the separation of bound from free detectable label needed to determine the results. Capture receptors may be trapped, for example, by mixing the receptors with a gel precursor and applying the mixture to some or a portion of the porous solid phase. The gel may then coalesce within the porous matrix and expose the capture receptors to fluids passing laterally through solid phase. Such entrapment methods are well known in the art. The region of the porous solid phase to which the capture receptor is applied may be formed as a graphical element so that, once a signal is developed, the graphical element presents an easy to interpret display of test results. The detection step in these assays depends on the reagents employed. Where the detectable label is a colored latex particle or a colloidal metal such as gold, the reaction result may be observed visually. The detectable label may alternatively include a fluor, or a florogen or lumogen (in embodiments where the signal is produced by action of an enzyme or catalytic label). These labels may be detected by appropriate instrumentation known in the art.

Another method of the invention uses small superparamagnetic particles as a solid phase. These particles may be mixed in a suspension containing the sample. The finely divided nature of the solid phase in such embodiments assures relatively short diffusion distances for the virions to encounter the capture ACE2. This may be useful for physically large analytes such as virions that diffuse slowly. The solACE2 may be labeled with an enzyme such as horseradish peroxidase or alkaline phosphatase. The particles may then be pulled to a location near a magnet so that they may be conveniently exposed to a relatively large volume of wash liquid by one or more repetitions of filling and draining the reaction vessel. Once the wash is complete, the particles may be resuspended and exposed to a chemiluminescent substrate of the enzyme. Many such substrates are commercially available, including Lumi-Phos HRP (PS-atto) acridan based chemiluminescent substrate for detection of horseradish peroxidase-conjugated molecules. This and similar materials for other enzymes are available from Lumigen of Southfield, Mich. This method advantageously allows for automation on existing fully automated platforms. In addition, the chemiluminescent detection process can afford high sensitivity. The detection step in these assays depends on the chemiluminescent (or electrochemiluminescent) reagents employed, but typically includes the known steps of collecting light emitted by the reaction mixture using a luminometer, a photodiode, an avalanche photodiode, a photomultiplier tube, a multi-pixel photon counter, an image sensor, or similar device known in the art.

In some embodiments it may be desirable to separate steps of combining the specimen with the solid phase and of adding a soluble receptor because the spike protein is being used for both immobilization and labeling. The soluble receptor may bind to some or most of the spike proteins, making capture by the solid phase less likely. Instead, the specimen may first be added to the solid phase and exposed for sufficient time (empirically determined for each particular embodiment, but likely in the few minutes range) to bind at least some of the virions. The soluble receptor may then be added and exposed for a time to the immobilized virions for sufficient time (to be empirically determined, but likely in the few minutes range) to bind at least some of the unoccupied spike proteins. Steric hindrance makes it unlikely that immobilized receptor will fully occupy all virion sites.

In some embodiments, the soluble receptor or the specimen may be suspended or mixed with in a wash liquid so that the binding of soluble receptor to spike proteins of nontarget coronavirus is limited by a more stringent binding conditions to favor the high affinity binding to SARS CoV-2. In other embodiments, the wash liquids may be added as a separate step.

In some embodiments, each assay may make two determinations to achieve a single result. In the first determination, washing with a low stringency wash liquid allows collection of a first signal that is a combination of signals attributable to virions having high affinity for the receptor and virions that have moderate affinity for the receptor. In the second determination, washing with a high stringency wash liquid allows collection of a second signal that is more highly weighted to virions having high affinity for the receptor. Comparison of the two signals (including combinations of the signals such as ratios between the two signals) indicates the presence of virions with high affinity binding. When high affinity binding is present, the test result may be treated as positive for the suspected infectious virus.

The embodiments described herein are referred in the specification as “one embodiment,” “an embodiment,” “another embodiment,” etc. These references indicate that the embodiment(s) described can include a particular feature, structure, or characteristic, but every embodiment does not necessarily include every described feature, structure, or characteristic. Further, when a particular feature, structure, or characteristic is described in connection with an embodiment, such feature, structure, or characteristic may also be used in connection with other embodiments whether or not explicitly described.

Further, where specific examples are given, the skilled practitioner may understand the particular examples as providing particular benefits such that the invention as illustratively disclosed herein suitably may be practiced in the absence of any element which is not specifically disclosed herein or within that particular example.

This disclosure cites or mentions certain other documents such as articles from the scientific literature and patents. Each of these documents is hereby incorporated by reference herein. Where such documents conflict with the express disclosure of this document, this document controls.

It will be apparent to those of ordinary skill in the art that many modifications and variations of the described embodiment are possible in the light of the above teachings without departing from the principles and concepts of the disclosure as set forth in the claims. Further, where specific examples are given, the skilled practitioner may understand the particular examples as providing particular benefits such that the invention as illustratively disclosed herein suitably may be practiced in the absence of any element which is not specifically disclosed herein or within that particular example.

Although the present disclosure describes certain exemplary embodiments, it is to be understood that such disclosure is purely illustrative and is not to be interpreted as limiting. Consequently, without departing from the spirit and scope of the disclosure, various alterations, modifications, and/or alternative applications of the disclosure will, no doubt, be suggested to those skilled in the art after having read the preceding disclosure. Accordingly, it is intended that the following claims be interpreted as encompassing all alterations, modifications, or alternative applications as fall within the true spirit and scope of the disclosure.

I claim:

1. A method of detecting a virus in a sample, the method comprising:
forming a reaction mixture by exposing an aliquot of a sample purportedly containing a virus to an immobilized receptor;
adding a soluble receptor including a detectable label;
washing the immobilized receptor; and
detecting a signal from the detectable label.

2. The method of claim 1, wherein the virus is a coronavirus.

3. The method of claim 1, wherein the immobilized receptor includes at least a portion of an angiotensin converting enzyme 2 (ACE2).

4. The method of claim 1, wherein the immobilized receptor includes a magnetic particle, a paramagnetic particle, a superparamagnetic particle, a dye-labeled particle, a porous substrate, a solid substrate, a gel, or a fluid interface.

5. The method of claim 4, wherein the solid substrate includes a wall of a container.

6. The method of claim 1, wherein the soluble receptor comprises at least a portion of an ACE2.

7. The method of claim 1, wherein the detectable label includes one or more of a dye, a fluorescent marker, a quantum dot, a mass tag, an enzyme, a nucleic acid, a particle, a magnetic tag, a radionuclide, or a colloidal metal.

8. The method of claim 1, wherein the step of washing the immobilized receptor includes exposing the immobilized receptor to a wash liquid.

9. The method of claim 8, wherein the step of washing the immobilized receptor includes controlled stringency conditions comprising control of one or more of a temperature, a pH, an ionic strength, a hydrophobicity, a chaotrope concentration, a surfactant concentration, a competitor concentration, a volume, or a flow rate of the wash liquid.

10. The method of claim 9, wherein the ionic strength is less than about 0.10 mol/kg.

11. The method of claim 9, further comprising:
forming a second reaction mixture by exposing a second aliquot of the sample to a second immobilized receptor;
adding a second soluble receptor including a second detectable label to the second reaction mixture;
washing the second immobilized receptor;
detecting a second signal from the second detectable label; and
comparing the signal to the second signal,
wherein the step of washing the second immobilized receptor occurs under different stringency conditions than the step of washing the immobilized receptor.

12. The method of claim 1, wherein the coronavirus is SARS-CoV-2, and wherein the immobilized receptor and the soluble receptor each include at least a portion of an ACE2.

13. The method of claim 12, wherein the immobilized receptor includes a superparamagnetic particle, and wherein the detectable label includes an enzyme capable of acting on a chemiluminescent substrate to produce a light.

14. The method of claim 12, wherein the immobilized receptor includes a lateral flow substrate.

15. The method of claim 12, wherein the immobilized receptor includes a particle and the step of detecting the signal includes characterizing the particle in a flow cytometer.

16. A kit comprising:
a lateral flow substrate;
an ACE2 immobilized to the lateral flow substrate;
a developing solution including an ACE2 coupled to a detectable label;
a first wash liquid.

17. The kit of claim 16, wherein the first wash liquid is configured to displace at least 50% of a SARS CoV coronavirus from the substrate but less than 50% of a SARS CoV-2 coronavirus from the substrate.

18. The kit of claim 16, further comprising a second wash liquid differing from the first wash liquid in one or more of a pH, an ionic strength, a hydrophobicity, a chaotrope concentration, a surfactant concentration, or a competitor concentration.

19. The kit of claim 16, wherein the first wash liquid has an ionic strength less than about 0.15 mol/kg, and wherein the first wash liquid includes an anionic surfactant or a chaotrope.

20. A kit comprising an immobilized ACE2, an ACE2 coupled to a detectable label, a first wash liquid having an ionic strength less than about 0.10 mol/kg, and a second wash liquid having an ionic strength greater than about 0.10 mol/kg.
